(19) United States
(12) Patent Application Publication
(10) Pub. No.: US 2010/0095396 A1
(43) Pub. Date: Apr. 15, 2010

(54) METHOD FOR INCREASING THE DRY BIOMASS OF PLANTS

(76) Inventors: Dirk Voeste, Limburgerhof (DE); Alissa Zeller, Singapore (SG); Egon Haden, Kleinniedesheim (DE)

Correspondence Address: BIRCH STEWART KOLASCH & BIRCH, PO BOX 747, FALLS CHURCH, VA, 22040-0747, US

(21) Appl. No.: 12/446,833

(22) PCT Filed: Nov. 16, 2007
(86) PCT No.: PCT/EP07/62463

§ 371 Date: Apr. 23, 2009

(30) Foreign Application Priority Data
Nov. 17, 2006 (EP) 06124358.0

(51) Int. Cl. A01H 1/06

(52) U.S. Cl. 800/276

(57) ABSTRACT

The present invention relates to a method for increasing the dry biomass of a plant via increasing the carbon assimilation by treating a plant, a part of the plant, the locus where the plant is growing or is intended to grow and/or the plant propagules with at least one insecticide. The invention also relates to a method for increasing the biomass of the fruit of a plant via increasing the carbon assimilation, the fruit containing 5 to 25% by weight of residual moisture, based on the total weight of the fruit, by treating a plant, a part of the plant, the locus where the plant is growing or is intended to grow and/or the plant propagules with at least one insecticide. The invention further relates to a method for increasing the carbon dioxide sequestration from the atmosphere by treating a plant, a part of the plant, the locus where the plant is growing or is intended to grow and/or the plant propagules with at least one insecticide as described below.

The present invention relates to a method for increasing the dry biomass of a plant by treating a plant, a part of the plant, the locus where the plant is growing or is intended to grow and/or the plant propagules with at least one insecticide. The invention also relates to a method for increasing the biomass of the fruit of a plant, the fruit containing 5 to 25% by weight of residual moisture, based on the total weight of the fruit, by treating a plant, a part of the plant, the locus where the plant is growing or is intended to grow and/or the plant propagules with at least one insecticide. The invention further relates to a method for increasing the carbon dioxide sequestration from the atmosphere by treating a plant, a part of the plant, the locus where the plant is growing or is intended to grow and/or the plant propagules with at least one insecticide as described below.

One of the biggest challenges to the world community in the coming years will be the reduction of gases responsible for the greenhouse effect in the atmosphere or at least the stabilization of greenhouse gas concentrations in the atmosphere at a level that would prevent dangerous anthropogenic interference with the climate system. Perhaps the most important of these greenhouse gases is carbon dioxide. This concern is expressed in the Kyoto Protocol in which the ratifying countries commit to reduce their emissions of carbon dioxide and five other greenhouse gases or engage in emissions trading if they maintain or increase emissions of these gases.

Atmospheric carbon dioxide originates from multiple natural sources including volcanic outgassing, the combustion of organic matter, and the respiration processes of living aerobic organisms. Anthropogenic carbon dioxide derives mainly from the combustion of various fossil fuels for power generation and transport use. Since the start of the Industrial Revolution, the atmospheric CO₂ concentration has increased by approximately 110 μl/l or about 40%, most of it released since 1945. Taking only into account the world's two biggest and fastest developing countries India and China, which make up for one third of the world population, and their estimated “energy hunger”, it can be expected that man-derived carbon dioxide output has by far not reached its culmination point. Alternative energy sources, such as solar, tidal or wind energy, are promising approaches but so far, they are neither effective nor flexible enough to replace energy from conventional combustion on a global scale. Since neither energy saving efforts nor alternative energy sources are likely to prevail in the next future, another approach to the reduction/stabilization of greenhouse gas concentration and thus to the compliance of the Kyoto Protocol becomes relevant: The sequestration of carbon dioxide from the atmosphere.

Main natural carbon dioxide sinks, i.e. carbon dioxide reservoirs preferably increasing in size, are oceans and growing vegetation.

Oceans represent probably the largest carbon dioxide sink on earth. This role as a sink for CO₂ is driven by two processes, the solubility pump and the biological pump. The former is primarily a function of differential CO₂ solubility in seawater and the thermohaline circulation, while the latter is the sum of a series of biological processes that transport carbon (in organic and inorganic forms) from the surface euphotic zone to the ocean's interior. A small fraction of the organic carbon transported by the biological pump to the seafloor is buried in anoxic conditions under sediments and ultimately forms fossil fuels such as oil and natural gas. However, little is known about the impact of climate modifications on the efficacy of the oceans as carbon sinks. For example, ocean acidification by invading anthropogenic CO₂ may affect the biological pump by negatively impacting calcifying organisms such as coccolithophores, foraminiferans and pteropods. Climate changes may also affect the biological pump by the future by warming and stratifying the surface ocean, thus reducing the supply of limiting nutrients to surface waters.

It therefore appears to be more promising to rely on vegetation as a carbon sink. This is also reflected in the Kyoto protocol, where countries having large areas of forest (or other vegetation) can deduct a certain amount from their emissions, thus making it easier for them to achieve the desired emission levels.

As part of photosynthesis, plants absorb carbon dioxide from the atmosphere. After metabolization, the produced carbohydrates are stored as sugar, starch and/or cellulose, while oxygen is released back to the atmosphere. In the soil, the gradual build-up of slowly decaying organic material accumulates carbon, too, thus forming a further carbon dioxide sink.

Forests are probably the most effective vegetative form of carbon sinks, but worldwide deforestation countervails this positive effect. Forests are mostly replaced by agricultural areas. Therefore, using agricultural vegetation as a carbon dioxide sink is a useful alternative. In this context, it is desirable to provide a method which makes plants increase their net uptake of carbon dioxide and their carbon assimilation in order to increase the amount of carbon dioxide sequestered from the atmosphere. An increased carbon assimilation generally involves an increased dry biomass of the plant or its crop.

Another major challenge to the world community in coming years will be keeping food production in pace with the increasing world population which is unfortunately accompanied by a worldwide decline of high quality arable land. Meeting this challenge will require efforts in multiple areas, one of which will be to provide crops with an increased nutritional value. The nutritional value is on the one side related with the biomass of the plant or of the crop. On the other side, the plant's or crop's biomass is also composed of water, so that a better measure is the dry biomass.

It is therefore an object of the present invention to provide a method for increasing the dry biomass of a plant, especially the dry carbon biomass.

Surprisingly, it was found that treating a plant and/or its locus of growth and/or its propagule with an insecticide leads to an enhanced carbon dioxide assimilation and thus to an enhanced dry biomass of the plant, especially to an enhanced dry carbon biomass.

Therefore, according to one aspect, the present invention provides a method for increasing the dry biomass of a plant via increasing the carbon dioxide assimilation which method comprises treating a plant, a part of the plant, the locus where the plant is growing or is intended to grow and/or the plant propagules with at least one insecticide.

The invention also relates to the use of insecticides for increasing the dry biomass, especially the dry carbon biomass, of a plant.

In the terms of the present invention, “biomass of a plant” is the total organic material produced by plants, such as leaves, roots, seeds, and stalks. Biomass is a complex mixture of organic materials, such as carbohydrates, fats and proteins, along with small amounts of minerals, such as sodium, calcium, iron and phosphorus. The main components of plant biomass are carbohydrates and lignin, the proportions of which vary with the plant type. “Biomass of a fruit” is the total mass of a fruit. The plant's or fruit's biomass also encompasses water contained in the plant/fruit tissue, if not specified otherwise.

In the terms of the present invention, “dry biomass” means the biomass of the plant after the plant has been dried to a residual moisture content of 0 to 1% by weight, preferably to a moisture content of 0 to 0.5% by weight and in particular to a moisture content of approximately 0% by weight. “Approximately” includes the standard error value. Drying can be carried out by any method suitable for drying the respective plant, for example, if necessary, first chopping the plant or parts thereof and then drying it in an oven, e.g. at 100° C. or more for an appropriate time. In one embodiment of the invention, the dry biomass of the total plant, i.e. including the roots, tuber, stem, leaves, fruits etc., is determined. This calculation base is preferably applied to tuber plants. In another embodiment, the dry biomass of the overground part of the plant, i.e. the plant without roots, tuber and other subterrestrial parts, is determined. To this end, the plant is capped tightly over the ground, dried and weighed. This calculation base is preferably applied to rooted plants (without tuber) yet since in some cases it is difficult to eradicate the plant together with the total root system. In yet another embodiment, the dry biomass of a predominant part of the plant, e.g. the leaves or the stem/stalk, is determined. In yet another embodiment, the dry biomass of the plant's crop is determined.

Propagules are all types of plant propagation material. The term embraces seeds, grains, fruit, tubers, rhizomes, spores, cuttings, offshoots, meristem tissues, single and multiple plant cells and any other plant tissue from which a complete plant can be obtained. One particular propagule is seed.

Locus means soil, area, material or environment where the plant is growing or intended to grow.

In another aspect, the invention relates to a method for increasing the biomass of the crop of a plant, the crop containing 0 to 25% by weight, preferably 0 to 16% by weight and more preferably 0 to 12% by weight of residual moisture (water), based on the total weight of the crop, which method comprises treating a plant, a part of the plant, the locus where the plant is growing or is intended to grow and/or the plant propagules with at least one insecticide.

The invention also relates to the use of at least one insecticide for increasing the biomass of the crop of a plant, the crop containing 0 to 25% by weight, preferably 0 to 16 and more preferably 0 to 12% by weight of residual moisture, based on the total weight of the crop.

“Crop” is to be understood as any plant product which is further utilized after harvesting, e.g. fruits in the proper sense, vegetables, nuts, grains, seeds, wood (e.g. in the case of silviculture plants), flowers (e.g. in the case of gardening plants, ornamentals) etc.; that means anything of economic value that is produced by the plant.

In yet another aspect, the invention relates to a method for increasing the biomass of the fruit of a plant, the fruit containing 5 to 25% by weight, preferably 8 to 16% by weight and more preferably 9 to 12% by weight of residual moisture (water), based on the total weight of the fruit, which method comprises treating a plant, a part of the plant, the locus where the plant is growing or is intended to grow and/or the plant propagules with at least one insecticide.

The invention also relates to the use of insecticides for increasing the biomass of the fruit of a plant, the fruit containing 5 to 25% by weight, preferably 8 to 16 or 9 to 12% by weight of residual moisture, based on the total weight of the fruit.

In the terms of the present invention, “fruit” is to be understood as any plant product which generally serves for the propagation of the plant, e.g. fruits in the proper sense, vegetables, nuts, grains or seeds.

The residual moisture of the crop or of the fruit can for example be determined by NIR (near infrared) spectroscopy or by electrical conductivity. Preferably, the crop or fruit is harvested at the point of time at which it has the proper water content. However, if this is not possible and the residual moisture content of the fruit or the crop is higher than the above values, the moisture content can be reduced by drying the crop or the fruit to the desired moisture content, e.g. by drying it in a drying oven. The moisture content can e.g. be then determined by comparing the weight of the dried fruit or crop with the weight before the drying process.

The increase in dry biomass is in particular based on an increase of the dry carbon biomass, which, in turn, is at least partly due to an increase of the carbon dioxide assimilation of the plant. While the method and the use according to the invention lead to a net increase of the carbon dioxide assimilation, at the same time the net photorespiration of the plant is reduced or is at least lower that the net increase of the carbon dioxide assimilation. “Net” refers to a value measured over the plant's lifetime. The increase in dry biomass is thus the result of an increased carbon dioxide sequestration from the atmosphere by a plant and is thus an increase of the dry carbon biomass. Carbon dioxide sequestration refers to carbon dioxide assimilation which is not annihilated by photorespiration.

Accordingly in yet another aspect, the invention relates to a method for increasing the carbon dioxide sequestration from the atmosphere by a plant which method comprises treating the plant, a part of the plant, the locus where the plant is growing or is intended to grow and/or the plant propagules with at least one insecticide.

The invention also relates to the use of insecticides for increasing the carbon dioxide sequestration from the atmosphere by a plant.

As already mentioned, it has to be emphasized that the increase in dry biomass, in the biomass of the fruit or crop and the increase in CO₂ sequestration are not only transitory effects but are net results over the whole lifetime of the plant or at least over an important part of the lifetime of the plant, for example until harvesting the plant, harvesting taking place at the point of time usual for the respective plant variety, or until the plant's death. Preferably, the increase in dry biomass of the plant or in biomass of the fruit/crop with the above-defined moisture content is determined after the plant has been harvested; harvesting taking place at the point of time usual for the respective plant variety.

The below remarks as to preferred embodiments of the insecticides, to their preferred use and to preferred embodiments of the methods of the invention are to be understood either each on their own or preferably in combination with each other.

Preferably, the at least one insecticide is selected from GABA antagonist compounds, nicotinic receptor agonist/antagonist compounds and anthranilamide compounds of formula Γ¹

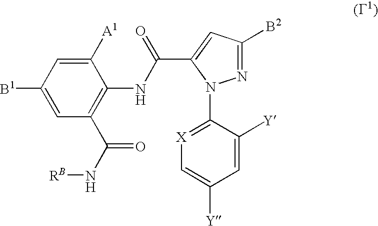

wherein

### A¹ is CH₃, Cl, Br or I;

### X is C—H, C—Cl, C—F or N;

### Y′ is F, Cl or Br;

### Y″ is F, Cl or CF₃;

B¹ is hydrogen, Cl, Br, I or CN;

### B² is Cl, Br, CF₃, OCH₂CF₃ or OCF₂H; and

R^{B} is hydrogen, CH₃ or CH(CH₃)₂.

Preferably, the GABA antagonist compounds are selected from acetoprole, endosulfan, ethiprole, 5-amino-1-(2,6-dichloro-α,α,α-trifluoro-p-tolyl)-4-trifluoromethylsulfinylpyrazole-3-carbonitrile (fipronil), vaniliprole, pyrafluprole, pyriprole and the phenylpyrazole compound of formula Γ²

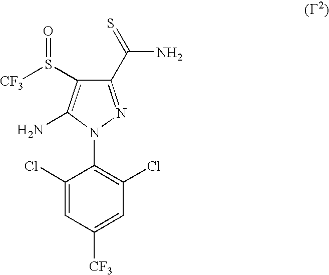

A particularly preferred GABA antagonist compound is 5-amino-1-(2,6-dichloro-α,α,α-trifluoro-p-tolyl)-4-trifluoromethylsulfinylpyrazole-3-carbonitrile, which is also known under the common name of fipronil.

Preferred nicotinic receptor agonist/antagonist compounds are selected from clothianidin, dinotefuran, (EZ)-1-(6-chloro-3-pyridylmethyl)-N-nitroimidazolidin-2-ylideneamine (imidacloprid), (EZ)-3-(2-chloro-1,3-thiazol-5-ylmethyl)-5-methyl-1,3,5-oxadiazinan-4-ylidene(nitro)amine (thiamethoxam), nitenpyram, acetamiprid and thiacloprid.

More preferred nicotinic receptor agonist/antagonist compounds are selected from imidacloprid, clothianidin and thiamethoxam.

In a more preferred embodiment, the insecticide is selected from GABA antagonist compounds

A particularly preferred insecticide is fipronil.

In one embodiment of the invention, more than one insecticide is used. For example, two or more different GABA antagonist compounds are used or two or more different nicotinic receptor agonist/antagonist compounds are used or two or more anthranilamide compounds of formula Γ¹ are used or one GABA antagonist compound is combined with another type of insecticide, e.g. a pyrethroide, or one nicotinic receptor agonist/antagonist compound is combined with another type of insecticide or one anthranilamide compound of formula Γ¹ is combined with another type of insecticide. In a specific embodiment, GABA antagonist compound, preferably fipronil, is combined with a pyrethroide insecticide. Preferred pyrethoide insecticides are selected from allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, gamma-cyhalothrin, permethrin, prallethrin, pyrethrin I and II, resmethrin, silafluofen, tau-fluvalinate, tefluthrin, tetramethrin, tralomethrin, transfluthrin and profluthrin, dimefluthrin, alpha-cypermethrin being particularly preferred.

The above insecticides and methods for producing them are generally known. For instance, the commercially available compounds may be found in The Pesticide Manual, 13^{th} Edition, British Crop Protection Council (2003) among other publications. Acetoprole and its preparation have been described in WO 98/28277. Pyrafluprole and its preparation have been described in JP 2002193709 and in WO 01/00614. Pyriprole and its preparation have been described in WO 98/45274 and in U.S. Pat. No. 6,335,357. Further insecticides can be prepared by methods analogous to those described in the above references.

If a part of the plant is to be treated by the method of the invention, e.g. the leaves, it is evident that the parts to be treated must be parts of a living plant, not of a harvested one. It is also evident that a plant to be treated is a living one.

In general, it is possible to use nearly all types of plants for the method of the present invention. However, taking into account economic considerations, the plants to be treated are preferably agricultural or silvicultural plants.

Agricultural plants are plants of which a part or all is harvested or cultivated on a commercial scale or serves as an important source of feed, food, fibers (e.g. cotton, linen), combustibles (e.g. wood, bioethanol, biodiesel, biomass) or other chemical compounds. Examples are soybean, corn (maize), wheat, triticale, barley, oats, rye, rape, such as canola, millet (sorghum), rice, sunflower, cotton, sugar beets, pome fruit, stone fruit, citrus, bananas, strawberries, blueberries, almonds, grapes, mango, papaya, peanuts, potatoes, tomatoes, peppers, cucurbits, cucumbers, melons, watermelons, garlic, onions, carrots, cabbage, beans, peas, lentils, alfalfa (lucerne), trefoil, clovers, flax, elephant grass (Miscanthus), grass, lettuce, sugar cane, tea, tobacco and coffee.

Silvicultural plants in the terms of the present invention are trees, more specifically trees used in reforestation or industrial plantations. Industrial plantations generally serve for the commercial production of forest products, such as wood, pulp, paper, rubber, Christmas trees, or young trees for gardening purposes. Examples for silviculturel plants are conifers, like pines, in particular Pinus spec., fir and spruce, eucalyptus, tropical trees like teak, rubber tree, oil palm, willow (Salix), in particular Salix spec., poplar (cottonwood), in particular Popolus spec., beech, in particular Fagus spec., birch and oak.

In one preferred embodiment, the agricultural plants are selected from plants which are suitable for (renewable) energy production. Preferred plants in this context are cereals, such as soybean, corn, wheat, barley, oats, rye, rape, millet and rice, sunflower and sugar cane. Specifically, the agricultural plants are selected from corn, soybean and sugar cane.

In another preferred embodiment, the agricultural plants are selected from legumes.

Legumes are particularly rich in proteins. Examples are all types of peas and beans, lentils, alfalfa (lucern), peanuts, trefoil, clovers and in particular soybeans.

In one preferred embodiment, the silvicultural plants are selected from eucalyptus, tropical trees like teak, rubber tree and oil palm tree, willow (Salix), in particular Salix spec., and poplar (cottonwood), in particular Popolus spec.

In one preferred embodiment, the plants are selected from plants which can be used in the production of (renewable) energy. Suitable plants in this context are oil plants, such as soybean, corn, oilseed rape (in particular canola), flax, oil palm, sunflower and peanuts. Further suitable plants are those for the production of bioethanol, such as sugar cane. Further suitable plants are those suitable for the production of biomass, such as all cereals from which the straw can be used as combustible biomass, e.g. soybean, corn, wheat, barley, oats, rye, rape, millet and rice, in particular corn, wheat, barley, oats, rye, rape, and millet, trees, in particular those having fast-growing wood, such as eucalyptus, poplar and willow, and also miscanthus. Preferred plants which can be used in the production of (renewable) energy are selected from soybean, corn, oilseed rape (in particular canola), flax, oil palm, peanuts, sunflower, wheat, sugar cane, eucalyptus, poplar, willow and miscanthus.

In another preferred embodiment, the plants are selected from starch-producing plants, preferably potato and cereals rich in starch, such as corn, wheat, barley, oats, rye, millet and rice, in particular potato and corn.

In another preferred embodiment, the plants are selected from plants suitable for the production of fibers, in particular cotton and flax.

In another preferred embodiment, the plants are selected from oil plants, such as soybean, corn, oilseed rape (in particular canola), flax, oil palm, sunflower and peanuts.

In another preferred embodiment, the plants are selected from monocotyledonous plants, such as corn, wheat, barley, oats, rye, millet, rice, bananas, garlic, onions, carrots, sugar cane and Miscanthus, in particular corn, wheat and Miscanthus.

In another preferred embodiment, the plants are selected from dicotyledonous plants, such as soybean, rape, sunflower, cotton, sugar beets, pome fruit, stone fruit, citrus, strawberries, blueberries, almonds, grapes, mango, papaya, peanuts, potatoes, tomatoes, peppers, cucurbits, cucumbers, melons, watermelons, cabbage, beans, peas, lentils, alfalfa (lucerne), trefoil, clovers, flax, elephant grass (Miscanthus), lettuce, tea, tobacco and coffee.

In a more preferred embodiment, however, the plants are selected from agricultural plants, which in turn are selected from soybeans and C4 plants, and from silvicultural plants, and even more preferably from C4 plants and silvicultural plants.

C4 plants are plants, which, when compared to C3 plants, have a faster photosynthesis under warm and light conditions and which have a further pathway for carbon dioxide fixation. In the simpler and more ancient C3 plants, the first step in the light-independent reactions of photosynthesis involves the fixation of CO₂ by the enzyme RuBisCo (ribulose bisphosphate carboxylase oxygenase; the first enzyme in the Calvin cycle) into 3-phosphoglyceric acid (PGA), a molecule with three carbon atoms (therefore “C3” plants), which serves as starting material for the synthesis of sugars and starch). However, due to the dual carboxylase/oxygenase activity of RuBisCo, an amount of the substrate is oxidized rather than carboxylated resulting in loss of substrate and consumption of energy in what is known as photorespiration. In order to bypass the photorespiration pathway, C4 plants have developed a mechanism to efficiently deliver CO₂ to the RuBisCO enzyme. They utilize their specific leaf anatomy where chloroplasts exist not only in the mesophyll cells in the outer part of their leaves but in the bundle sheath cells as well. Instead of direct fixation in the Calvin cycle, CO₂ is converted to an organic acid with four carbon atoms (therefore “C4”) which has the ability to regenerate CO₂ in the chloroplasts of the bundle sheath cells. Bundle sheath cells can then utilize this CO₂ to generate carbohydrates by the conventional C3 pathway. C4 plants are superior to C3 plants as regards their water-use-efficiency (WUE), i.e. they need less water for the formation of the same dry mass. Most known C4 plants are grasses, followed by sedges.

In the terms of the present invention, preferred C4 plants are selected from corn, sugar cane, millet, sorghum, elephant grass (Miscanthus), switchgrass (Miscanthus sinensis) and amaranth.

Specifically, the plants are selected from corn and sugar cane and more specifically from corn.

Preferred crops are grains, in particular cereal grains, such as soybean, corn, wheat, triticale, barley, oats, rye, rape, millet, and rice grains, further sunflower grains, cotton grains and peanuts, straw, in particular from cereals such as corn, wheat, triticale, barley, oats, rye, rape and millet, or from miscanthus, and wood, in particular from fast-growing trees, such as eucalyptus, poplar and willow. More preferred crops are grains and straw.

The plants can be non-transgenic plants or can be plants that have at least one transgenic event. In case the insecticides used according to the invention are used together with another pesticide, e.g. a herbicide, in one embodiment it is preferred that the plant be a transgenic plant having preferably a transgenic event that confers resistance to the particular pesticide. For example, if the additional pesticide is the herbicide glyphosate, it is preferred that the transgenic plant or propagules be one having a transgenic event that provides glyphosate resistance. Some examples of such preferred transgenic plants having transgenic events that confer glyphosate resistance are described in U.S. Pat. No. 5,914,451, U.S. Pat. No. 5,866,775, U.S. Pat. No. 5,804,425, U.S. Pat. No. 5,776,760, U.S. Pat. No. 5,633,435, U.S. Pat. No. 5,627,061, U.S. Pat. No. 5,463,175, U.S. Pat. No. 5,312,910, U.S. Pat. No. 5,310,667, U.S. Pat. No. 5,188,642, U.S. Pat. No. 5,145,783, U.S. Pat. No. 4,971,908 and U.S. Pat. No. 4,940,835. When the transgenic plant is a transgenic soybean plant, such plants having the characteristics of “Roundup-Ready” transgenic soybeans (available from Monsanto Company, St. Louis, Mo.) are preferred.

It is to be understood, however, that when the plant is a transgenic plant, the transgenic events that are present in the plant are by no means limited to those that provide pesticide resistance, but can include any transgenic event. In fact, the use of “stacked” transgenic events in a plant is also contemplated.

In one embodiment of the invention, the insecticides are used together with at least one further pesticide. Suitable pesticides are for example herbicides, such as the above-mentioned glyphosate, and fungicides.

The treatment of a plant or propagation material, such as a seed, with the at least one insecticide by the method of this invention can be accomplished in several ways. The agent (optionally together with one or more of the above additional pesticides) may be applied directly to the propagules, especially the seed, and/or to soil in which the seed is to be planted, for example, at the time of planting along with the seed (for example in-furrow application). Alternatively, it may be applied to the soil after planting and germination, or to the foliage of the plant after emergence and/or during the whole life cycle of the plant.

In ready-to-use preparations, the at least one insecticide can be present in suspended, emulsified or dissolved form. The application forms depend entirely on the intended uses.

The at least one insecticide can be applied as such, in the form of its formulations or the application form prepared therefrom, for example in the form of directly sprayable solutions, powders, suspensions or dispersions, including highly concentrated aqueous, oily or other suspensions or dispersions, emulsions, oil dispersions, pastes, dusts, compositions for broadcasting or granules. Application is usually by spraying, atomizing, dusting, broadcasting or watering. The application forms and methods depend on the intended uses; in each case, they should ensure the finest possible distribution of the active compounds.

Depending on the embodiment in which the ready-to-use preparations of the at least one insecticide is present, they comprise one or more liquid or solid carriers, if appropriate surfactants and if appropriate further auxiliaries customary for formulating crop protection agents. The recipes for such formulations are familiar to the person skilled in the art.

Aqueous application forms can be prepared, for example, from emulsion concentrates, suspensions, pastes, wettable powders or water-dispersible granules by addition of water. To prepare emulsions, pastes or oil dispersions, the active compounds, as such or dissolved in an oil or solvent, can be homogenized in water by means of a wetting agent, tackifier, dispersant or emulsifier. However, it is also possible to prepare concentrates composed of active substance, wetting agent, tackifier, dispersant or emulsifier and, if appropriate, solvent or oil, such concentrates being suitable for dilution with water.

The concentrations of the at least one insecticide in the ready-to-use preparations can be varied within relatively wide ranges. In general, they are between 0.0001 and 10%, preferably between 0.01 and 1% (% by weight total content of active compound, based on the total weight of the ready-to-use preparation).

The at least one insecticide may also be used successfully in the ultra-low-volume process (ULV), it being possible to employ formulations comprising more than 95% by weight of active compound, or even to apply the active compounds without additives.

Oils of various types, wetting agents, adjuvants, herbicides, fungicides, insecticides different from the at least one insecticide used according to the invention, nematicides, other pesticides, such as bactericides, fertilizers and/or growth regulators may be added to the active compounds, even, if appropriate, not until immediately prior to use (tank mix). These agents can be mixed in a weight ratio of from 1:100 bis 100:1, preferably from 1:10 to 10:1 with the at least one insecticide employed according to the invention.

Adjuvants are for example: modified organic polysiloxanes, e.g. Break Thru S 240®; alkohol alkoxylates, e.g. Atplus 245®, Atplus MBA 1303®, Plurafac LF 300® and Lutensol ON 30®; EO-PO block copolymers, e.g. Pluronic RPE 2035® and Genapol B®; alkohol ethoxylates, e.g. Lutensol XP 80®; and sodium dioctylsulfosuccinate, e.g. Leophen RA®.

The formulations are prepared in a known manner, for example by extending the active compounds with solvents and/or carriers, if desired with the use of surfactants, i.e. emulsifiers and dispersants. Solvents/carriers suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, methyl hydroxybutyl ketone, diacetone alcohol, mesityl oxide, isophorone), lactones (for example gamma-butyrolactone), pyrrolidones (pyrrolidone, N-methylpyrrolidone, N-ethylpyrrolidone, n-octylpyrrolidone), acetates (glycol diacetate), glycols, dimethyl fatty acid amides, fatty acids and fatty acid esters. In principle, solvent mixtures may also be used.
Carriers such as ground natural minerals (for example kaolins, clays, talc, chalk) and ground synthetic minerals (for example finely divided silica, silicates); emulsifiers such as nonionic and anionic emulsifiers (for example polyoxyethylene fatty alcohol ethers, alkylsulfonates and arylsulfonates), and dispersants such as lignosulfite waste liquors and methylcellulose.

Suitable surfactants are alkali metal salts, alkaline earth metal salts and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenyl polyglycol ether, tributylphenyl polyglycol ether, tristerylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable and animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, mesityl oxide, isophorone, strongly polar solvents, for example dimethyl sulfoxide, 2-yrrolidone, N-methylpyrrolidone, butyrolactone, or water.

Powders, compositions for broadcasting and dusts can be prepared by mixing or jointly grinding the active substances with a solid carrier.

Granules, for example coated granules, impregnated granules and homogeneous granules, can be prepared by binding the active compounds onto solid carriers. Solid carriers are, for example, mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers such as, for example, ammonium sulfate, ammonium phosphate, ammonium nitrate, ureas and plant products such as cereal meal, tree bark meal, wood meal and nutshell meal, cellulose powder and other solid carriers.

Formulations for seed treatment can further comprise binders and/or gelling agents and optionally colorants.

In general, the formulations comprise between 0.01 and 95% by weight, preferably between 0.1 and 90% by weight, in particular 5 to 50% by weight, of the active compound. In this context, the active compounds are employed in a purity of from 90% to 100%, preferably 95% to 100% (according to NMR spectrum).

After two- to ten-fold dilution, formulations for seed treatment comprise 0.01 to 60% by weight, preferably 0.1 to 40% by weight of the active compounds in the ready-to-use preparations.

Examples of formulations are:

### 1. Products for Dilution in Water

I) Water-Soluble Concentrates (SL, LS)

10 parts by weight of active compound are dissolved in 90 parts by weight of water or a water-soluble solvent. Alternatively, wetting agents or other adjuvants are added. Upon dilution in water, the active compound dissolves. The ready formulation contains 10% by weight of active ingredient.

II) Dispersible Concentrates (DC)

20 parts by weight of active compound are dissolved in 70 parts by weight of cyclohexanone with addition of 10 parts by weight of a dispersant, for example polyvinylpyrrolidone. The active ingredient is contained in 20% by weight. Upon dilution in water, a dispersion results.

III) Emulsifiable Concentrates (EC)

15 parts by weight of active compound are dissolved in 75 parts by weight of xylene with addition of calcium dodecylbenzenesulfonate and castor oil ethoxylate (in each case 5 parts by weight). The active ingredient is contained in 15% by weight. Upon dilution in water, an emulsion results.

IV) Emulsions (EW, EO, ES)

25 parts by weight of active compound are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier (Ultraturrax) and made into a homogeneous emulsion. The active ingredient is contained in 25% by weight. Upon dilution in water, an emulsion results.

V) Suspensions (SC, OD, FS)

20 parts by weight of active compound are comminuted in a stirred ball mill with addition of 10 parts by weight of dispersants, wetting agents and 70 parts by weight of water or an organic solvent to give a fine suspension of active compound. The active ingredient is contained in 20% by weight. Upon dilution in water, a stable suspension of the active compound results.

VI) Water-Dispersible and Water-Soluble Granules (WG, SG)

50 parts by weight of active compound are ground finely with addition of 50 parts by weight of dispersants and wetting agents and made into water-dispersible or water-soluble granules by means of technical apparatuses (for example extrusion, spray tower, fluidized bed). The active ingredient is contained in 50% by weight. Upon dilution in water, a stable dispersion or solution of the active compound results.

VII) Water-Dispersible and Water-Soluble Powders (WP, SP, SS, WS)

75 parts by weight of active compound are ground in a rotor-stator mill with addition of 25 parts by weight of dispersants, wetting agents and silica gel. The active ingredient is contained in 75% by weight. Upon dilution in water, a stable dispersion or solution of the active compound results.

VIII) Gel Formulations (GF)

20 parts by weight of active compound, 10 parts by weight of dispersants, 1 part by weight of gelling agent and 70 parts by weight of water or an organic solvent are ground in a ball mill to give a finely divided suspension. Upon dilution in water, a stable suspension of the active compound results.

### 2. Products for Direct Application

IX) Dusts (DP, DS)

5 parts by weight of active compound are ground finely and mixed intimately with 95 parts by weight of finely particulate kaolin. This gives a dust with 5% by weight of active ingredient.

X) Granules (GR, FG, GG, MG)

0.5 part by weight of active compound is ground finely and combined with 95.5 parts by weight of carriers. Current methods are extrusion, spray drying or the fluidized bed. This gives granules for direct application with 0.5% by weight of active ingredient.

XI) ULV Solutions (UL)

10 parts by weight of active compound are dissolved in 90 parts by weight of an organic solvent, for example xylene. This gives a product for direct application with 10% by weight of active ingredient.

Formulations suitable for treating seed are, for example:

I soluble concentrates (SL, LS)
III emulsifiable concentrates (EC)
IV emulsions (EW, EO, ES)
V suspensions (SC, OD, FS)
VI water-dispersible and water-soluble granules (WG, SG)
VII water-dispersible and water-soluble powders (WP, SP, WS)
VIII gel formulations (GF)
IX dusts and dust-like powders (DP, DS)

Preferred formulations to be used for seed treatment are FS formulations. Generally, theses formulations comprise 1 to 800 g/l of active compounds, 1 to 200 g/l of wetting agents, 0 to 200 g/l of antifreeze agents, 0 to 400 g/l of binders, 0 to 200 g/l of colorants (pigments and/or dyes) and solvents, preferably water.

Preferred FS formulations of the active compounds for the treatment of seed usually comprise from 0.5 to 80% of active compound, from 0.05 to 5% of wetting agent, from 0.5 to 15% of dispersant, from 0.1 to 5% of thickener, from 5 to 20% of antifreeze agent, from 0.1 to 2% of antifoam, from 1 to 20% of pigment and/or dye, from 0 to 15% of tackifier or adhesive, from 0 to 75% of filler/vehicle, and from 0.01 to 1% of preservative.

Suitable pigments or dyes for formulations of the active compounds for the treatment of seed are Pigment blue 15:4, Pigment blue 15:3, Pigment blue 15:2, Pigment blue 15:1, Pigment blue 80, Pigment yellow 1, Pigment yellow 13, Pigment red 112, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Suitable wetting agents and dispersants are in particular the surfactants mentioned above. Preferred wetting agents are alkylnaphthalenesulfonates, such as diisopropyl- or diisobutylnaphthalenesulfonates. Preferred dispersants are nonionic or anionic dispersants or mixtures of nonionic or anionic dispersants. Suitable nonionic dispersants are in particular ethylene oxide/propylene oxide block copolymers, alkylphenol polyglycol ethers and also tristryrylphenol polyglycol ether, for example polyoxyethylene octylphenol ether, ethoxylated isooctylphenol, octylphenol, nonylphenol, alkylphenol polyglycol ethers, tributylphenyl polyglycol ether, tristerylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters and methylcellulose. Suitable anionic dispersants are in particular alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore arylsulfonate/formaldehyde condensates, for example condensates of sulfonated naphthalene and naphthalene derivatives with formaldehyde, condensates of naphthalene or of naphthalenesulfonic acid with phenol and formaldehyde, lignosulfonates, lignosulfite waste liquors, phosphated or sulfated derivatives of methylcellulose and polyacrylic acid salts.

Suitable for use as antifreeze agents are, in principle, all substances which lower the melting point of water. Suitable antifreeze agents include alkanols, such as methanol, ethanol, isopropanol, the butanols, glycol, glycerol, diethylene glycol and the like.

Suitable thickeners are all substances which can be used for such purposes in agrochemical compositions, for example cellulose derivatives, polyacrylic acid derivatives, xanthane, modified clays and finely divided silica.

Suitable for use as antifoams are all defoamers customary for formulating agrochemically active compounds. Particularly suitable are silicone antifoams and magnesium stea rate.

Suitable for use as preservatives are all preservatives which can be employed for such purposes in agrochemical compositions. Dichlorophene, isothiazolenes, such as 1,2-benzisothiazol-3(2H)-one, 2-methyl-2H-isothiazol-3-one hydrochloride, 5-chloro-2-(4-chlorobenzyl)-3(2H)-isothiazolone, 5-chloro-2-methyl-2H-isothiazol-3-one, 5-chloro-2-methyl-2H-isothiazol-3-one, 5-chloro-2-methyl-2H-isothiazol-3-one hydrochloride, 4,5-dichloro-2-cyclohexyl-4-isothiazolin-3-one, 4,5-dichloro-2-octyl-2H-isothiazol-3-one, 2-methyl-2H-isothiazol-3-one, 2-methyl-2H-isothiazol-3-one calcium chloride complex, 2-octyl-2H-isothiazol-3-one, and benzyl alcohol hemiformal may be mentioned by way of example.

Adhesives/tackifiers are added to improve the adhesion of the effective components on the seed after treating. Suitable adhesives are EO/PO-based block copolymer surfactants, but also polyvinyl alcohols, polyvinyl pyrrolidones, polyacrylates, polymethacrylates, polybutenes, polyisobutenes, polystyrene, polyethyleneamines, polyethyleneamides, polyethyleneimines (Lupasol®, Polymin®), polyethers and copolymers derived from these polymers.

Suitable compositions for soil treatment include granules which may be applied in-furrow, as broadcast granules or as impregnated fertilizer granules, and also spray applications which are applied to the soil as a preemergent or postemergent spray.

Suitable compositions for treating the plants, in particular the overground parts thereof, especially the leaves (=foliar application) include spray applications, dusts and microgranules, spray applications being preferred.

Formulations suitable for producing spray solutions for the direct application are:

I soluble concentrates (SL, LS)
II) dispersible concentrates (DC)
III emulsifiable concentrates (EC)
IV emulsions (EW, EO)
V suspensions (SC)
VI water-dispersible and water-soluble granules (WG)
VII water-dispersible and water-soluble powders (WP, SP)

The methods of the invention are generally carried out by bringing the plant to be treated, parts of plant, the locus where the plant is growing or is intended to grow and/or its propagules in contact with the at least one insecticide or with a composition/formulation comprising it. To this end, the composition or the individual active compounds are applied to the plant, parts of plant, the locus where the plant is growing or is intended to grow and/or its propagules.

For treating the seed, it is possible in principle to use any customary methods for treating or dressing seed, such as, but not limited to, seed dressing, seed coating, seed dusting, seed soaking, seed film coating, seed multilayer coating, seed encrusting, seed dripping, and seed pelleting. Specifically, the treatment is carried out by mixing the seed with the particular amount desired of seed dressing formulations either as such or after prior dilution with water in an apparatus suitable for this purpose, for example a mixing apparatus for solid or solid/liquid mixing partners, until the composition is distributed uniformly on the seed. If appropriate, this is followed by a drying operation.

For treating the locus where the plant is growing or intended to grow, especially the soil, the latter may be treated by applying to the soil before the propagule is planted/sowed, at the time of planting or sowing along with the propagule (in case of seed sowing this is called in-furrow application), after planting/sowing or even after germination of the plant with a suitable amount of a formulation of the at least one insecticide either as such or after prior dilution with water.

Soil application is for example a suitable method for cereals, cotton, sunflower and trees, in particular if growing in a plantation.

Treatment of the plants or of overground parts thereof, especially their leaves, is in general carried out by spraying the plant or the overground parts thereof, especially their leaves (=foliar application) with a spraying liquor containing the active compound(s) or a formulation thereof in diluted or finely dispersed form. Application can be carried out, for example, by customary spray techniques using spray liquor amounts of from about 100 to 1000 l/ha (for example from 300 to 400 l/ha) using water as carrier. Application of the active compounds by the low-volume and ultra-low-volume method is possible, as is their application in the form of microgranules.

The required application rate of pure insecticide, i.e. active compound without formulation auxiliaries, depends on the composition of the plant stand, on the development stage of the plants, on the climatic conditions at the application site and on the application method. In general, the amount of compound applied is from 0.001 to 3 kg/ha, preferably from 0.005 to 2 kg/ha and in particular from 0.01 to 1 kg/ha of active substance (a.s.).

In the treatment of seed, the amount of active compound used is from 0.1 g to 10 kg per 100 kg of seed, preferably from 1 to 2.5 kg per 100 kg and mor preferably 1 to 200 g/100 kg, in particular from 5 to 100 g/100 kg. For specific crops such as lettuce and onions the rates can be higher.

The at least one insecticide is applied to the plants and/or the locus where the plants are growing or are intended to grow 1 to 10 times per season, preferably 1 to 5 times, more preferably 1 to 3 times and in particular 1 or 2 times per season.

Treatment of the propagules is in general only suitable for annual plants, i.e. for plants which are completely harvested after one season and which have to be replanted for the next season.

In one preferred embodiment, in the methods of the present invention, the propagules, especially the seeds, and/or the soil where the plants grow, preferably the propagules, especially the seeds, and/or the soil where the plants grow, are treated with the at least one insecticide. More preferably, the propagules, especially the seeds, are treated with the at least one insecticide.

In case of soil treatment and in particular of foliar treatment, the soil or the plants are treated after emergence of the plant. Preferably, the plants are treated in the growing stage 30 to 70 (according to the BBCH (Biologische Bundesanstalt für Land- und Forstwirtschaft, Bundessortenamt und Chemische Industrie (Federal Office for agriculture and silviculture, Republic of Germany) extended scale (a system for a uniform coding of phonologically similar growth stages of all mono- and dicotyledonous plant species; see www.bba.de/veroeff/bbch/bbcheng.pdf), i.e. from stem elongation or rosette growth/development of main shoot until flowering. The optimum time for treatment depends on the specific plant species and can easily be determined by appropriate tests.

By the methods of the invention, the dry biomass of plants and/or the biomass of the plant's crop having a moisture content of from 0 to 25% by weight, preferably of from 0 to 16% by weight and more preferably of from 0 to 12% by weight, based on the total weight of the crop, and/or the biomass of the plant's fruits having a moisture content of from 5 to 25% by weight, preferably of from 8 to 16% by weight and more preferably of from 9 to 12% by weight, based on the total weight of the fruit, is increased as compared to plants which have been grown under the same conditions but without being treated according to the invention and as compared to their crops/fruits having a comparable water content. This means that the treated plants have a better carbon assimilation and optionally also a better nitrogen assimilation, as compared to plants not treated according to the invention.

On the one hand, a better carbon assimilation is directly related with an increased carbon dioxide sequestration from the air because carbon dioxide is the essential source of carbohydrates in plants. This means that treating plants or parts thereof or the growth locus or plant propagules with insecticides used according to the invention leads to an enhanced net uptake of carbon dioxide by the plant, i.e. to an enhanced CO₂ sequestration from the atmosphere as compared to untreated plants. Sequestered CO₂ is not completely emitted again by the plant, as is proved by the enhanced C assimilation reflected in an increased dry biomass of the plant and/or biomass of the plant's fruits at a given moisture content. By this effect, the role of growing vegetation as a CO₂ sink can significantly be improved. An enhanced CO₂ net uptake means an improved CO₂ balance in the terms of the Kyoto Protocol.

On the other hand, a better carbon and nitrogen assimilation is related with an enhanced nutritional value of the plant or of the parts thereof used for food and feeds.

Without wishing to be bound by theory, it is supposed that one of the factors which contribute to an increased CO₂ sequestration and an increased carbon assimilation in the plant is that the insecticides used according to the invention lead to a decreased respiration of the plant and thus to a reduced carbon loss by CO₂ release during respiration. The decreased respiration is not a transitory effect, but is probably more or less continuously present during the whole or at least during an important part of the lifetime of the plant. It is further supposed that an increased nitrogen assimilation in the plant, which may additionally take place, is due to an enhanced nitrate reductase activity caused directly or indirectly by the insecticides used according to the present invention. It is further supposed that the insecticides used according to the invention also induce an enhanced tolerance of the plant toward abiotic stress such as temperature extremes, drought, extreme wetness or radiation, thus improving the plant's ability to store energy (carbohydrates, proteins, and thus dry biomass) even under unfavorable conditions. There are probably further factors which contribute to an enhanced C and N assimilation.

It has to be emphasized that the above effects of the insecticides used according to the invention, i.e. enhanced dry biomass of the plant, enhanced biomass of the fruit having the above specified moisture content, and increased CO₂ sequestration from the atmosphere also are present when the plant is not under biotic stress and in particular when the plant is not under pest pressure. It is evident that a plant suffering from pest (insect) attack produces a smaller biomass and a smaller crop yield as compared to a plant which has been subjected to curative or preventive treatment against the harmful insect and which can grow without the damage caused by the pest. However, the methods according to the invention leave to an enhanced dry biomass of the plant, an enhanced biomass of the fruit having the above specified moisture content, and/or to an increased CO₂ sequestration from the atmosphere by the plant even in the absence of any biotic stress and in particular of any deleterious pest. This means that the positive effects of the insecticides used according to the invention cannot be explained just by the insecticidal activities of these compounds, but are based on further activity profiles. But of course, plants under pest stress can be treated, too, according to the methods of the present invention.

The following examples shall further illustrate the invention without limiting it.

### EXAMPLES

### 1. Increase of the Dry Biomass of Corn Plants

Treatment with fipronil or fipronil+alpha-cypermethrin under free disease conditions

Corn was cultivated under customary conditions at Campinas (Brazil) in 2005/2006. A part of the seeds of the test plants had beforehand been treated with fipronil. Another part of the seeds of the plants had beforehand been treated with a mixture of fipronil and alpha-cypermethrin. 37 days after the emergence of the plants, the plants and their roots were harvested. The leaves and the roots were completely dried in an oven at 110° C. and then weighed. The results are compiled below.

| | | | Fipronil + alpha- |
|---|---|---|---|
| Treatment | Control | Fipronil | cypermethrin |
| Dry weight leaves | 20.91 | 23.38 | 24.5 |
| Dry weight roots | 34.25 | 41.27 | 45.18 |

As can be seen, the dry biomass of both the corn leaves and the corn roots is significantly increased by the treatment according to the invention as compared to untreated plants.

### 2. Increase of the Dry Biomass of Soybean Plants

Treatment with fipronil or fipronil+alpha-cypermethrin under free disease conditions

Soybean was cultivated under customary conditions at Campinas (Brazil) in 2005/2006. A part of the seeds of the test plants had beforehand been treated with fipronil. Another part of the seeds of the plants had beforehand been treated with a mixture of fipronil and alpha-cypermethrin. 35 days after the emergence of the plants, the plants and their roots were harvested. The leaves and the roots were completely dried in an oven at 110° C. and then weighed. The results are compiled below.

| | | | Fipronil + alpha- |
|---|---|---|---|
| Treatment | Control | fipronil | cypermethrin |
| Dry weight leaves | 9.92 | 13.15 | 11.10 |
| Dry weight roots | 7.58 | 15.6 | 11.69 |

As can be seen, the dry biomass of both the soybean leaves and the soybean roots is significantly increased by the treatment according to the invention as compared to untreated plants.

1-16. (canceled)

17. A method for increasing the dry biomass of a plant via increasing the carbon assimilation which method comprises treating a plant, a part of the plant, the locus where the plant is growing or is intended to grow and/or the plant propagules with at least one insecticide.

18. The method as claimed in claim 17, where the insecticide is selected from GABA antagonist compounds, nicotinic receptor agonist/antagonist compounds and anthranilamide compounds of formula Γ¹ 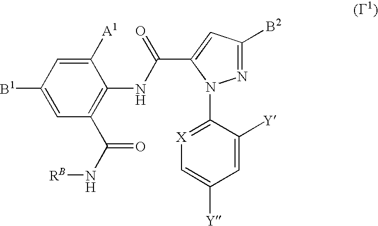
wherein
A¹ is CH₃, Cl, Br or I;
X is C—H, C—Cl, C—F or N;
Y′ is F, Cl or Br;
Y″ is F, Cl or CF₃;
B¹ is hydrogen, Cl, Br, I or CN;
B² is Cl, Br, CF₃, OCH₂CF₃ or OCF₂H; and
R^{B} is hydrogen, CH₃ or CH(CH₃)₂.

19. The method as claimed in claim 18, where the GABA antagonist compounds are selected from acetoprole, endosulfan, ethiprole, 5-amino-1-(2,6-dichloro-α,α,α,α-trifluoro-p-tolyl)-4-trifluoromethylsulfinylpyrazole-3-carbonitrile (fipronil), vaniliprole, pyrafluprole, pyriprole and the phenylpyrazole compound of formula Γ²

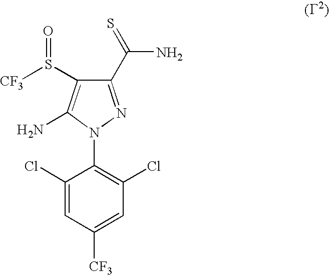

20. The method as claimed in claim 19, where the GABA antagonist compound is fipronil.

21. The method as claimed in claim 18, where the nicotinic receptor agonist/antagonist compounds are selected from clothianidin, dinotefuran, (EZ)-1-(6-chloro-3-pyridylmethyl)-N-nitroimidazolidin-2-ylideneamine (imidacloprid), (EZ)-3-(2-chloro-1,3-thiazol-5-ylmethyl)-5-methyl-1,3,5-oxadiazinan-4-ylidene(nitro)amine (thiamethoxam), nitenpyram, acetamiprid and thiacloprid.

22. The method as claimed in claim 21, where the nicotinic receptor agonist/antagonist compounds are selected from imidacloprid, thiamethoxam and clothianidin.

23. The use of an insecticide as defined in claim 17 for increasing the carbon dioxide sequestration from the atmosphere by a plant, where the plant is selected from soybean and C4 plants.

24. The method or use as claimed in claim 17, where the C4 plants are selected from corn, sugar cane, millet, sorghum, elephant grass (Miscanthus), switchgrass (Miscanthus sinensis) and amaranth.
